(19) United States
(12) Patent Application Publication
(10) Pub. No.: US 2021/0395820 A1
(43) Pub. Date: Dec. 23, 2021

(54) METHOD OF EVALUATING FEMALE REPRODUTIVE FUNCTION

(71) Applicants: UNIVERSIDADE DO PORTO, Porto (PT); UNIVERSIDADE DE AVEIRO, Aveiro (PT); CENTRO HOSPITALAR DO PORTO, E.P.E., Porto (PT)

(72) Inventors: Paula Maria VIEIRA JORGE, Leca Do Blio (PT); Antonio Jose ARSENIA NOGUEIRA, Aveiro (PT); Barbara Luisa CERQUEIRA RODRIGUES, Caldelas (PT)

(21) Appl. No.: 17/293,281

(22) PCT Filed: Dec. 6, 2019
(86) PCT No.: PCT/IB2019/060520

§ 371 Date: May 12, 2021

(30) Foreign Application Priority Data
Jan. 4, 2019 (PT) 115244

(51) Int. Cl. C12Q 1/6876

(52) U.S. Cl. CPC C12Q 1/6876; G01N 2800/367; C12Q 2537/165

(57) ABSTRACT

A non-invasive method to evaluate the reproductive function in female subjects is disclosed. The method disclosed herein provides assessing female reproductive function and ovarian response based on the number of CGG repeats and AGG interspersion number and pattern on each of the FMR1 gene alleles. Using a mathematical formula, it is possible to calculate an allelic score that differentiates those subjects with a better reproductive performance. This solution can thus be used routinely as a biomarker for predicting infertility or in the selection of ideal ovarian donor candidates.

### TECHNICAL FIELD

The present application relates to a method of evaluating female reproductive function.

### BACKGROUND ART

The influence of Fragile mental retardation-1 gene (FMR1) expansions on the female reproductive function was first recognized in carriers of premutations (CGG number between 55 and 200) which augmented the risk of Fragile X-associated primary ovarian insufficiency (FXPOI; OMIM #311360), in about 15 to 20%, when compared with full mutation carriers (CGGs above 200) (1,2). This condition is characterized by reduced function of the ovaries and accounts for about 5% of all cases of primary ovarian insufficiency (3). FXPOI can cause early menopause (below 35 years of age), irregular cycles, elevated follicle stimulating hormone (FSH) levels and ultimately lead to infertility (4,5).

Below 54 CGG repeats, alleles are classified as normal and usually carry two or more AGG interruptions which is assumed to give stability hampering the expansion to pathogenic ranges (6,7). A sub-genotype of normal alleles, with 45 to 54 CGGs, known as intermediate or gray zone, was defined, due to the likelihood of expansion (in two or three generations) (8,9). When this expansion is in full mutation range the hypermethylation of this repetitive region as well as the FMR1 promoter, lead to gene silencing. FMR1 transcriptional inactivation and the consequent absence of the coded protein, FMRP, is the cause of the intellectual disability in patients with fragile X syndrome [FXS; OMIM #300624] (9,10). FXS includes also learning problems, autistic behaviour and typical physical features, such as long and narrow face and protruding ears (9,11). FMRP plays a role in the development of connections at synapses (10,12). Although arising from mutations in the same gene, different mechanisms lead to FXS and FXPOI (4). The FMRP implication in the ovarian function, remains to be unravelled, although it is established that the premutation triggers the overproduction of FMR1 mRNA that leads to a process of RNA toxicity (2,13,14).

Recent reporting of phenotypes that overlap to those seen in females with premutations or are exclusive to normal/intermediate size carriers has grown interest in this latter range of alleles. Conclusions however, are controversial, not only regarding influence of FMR1 in ovarian reserve but also on definition of a “new” normal repeat range applicable exclusively in the female reproductive function. Gleicher, and co-workers (2015), published several studies showing the influence of the CGG repeat number in the ovarian reserve. In another study, an AMH decline, suggestive of diminished ovarian reserve, was observed to occur more rapidly in oocyte donor candidates carrying one allele with a CGG number below 26 (15). In a cohort of infertile women, lower AMH levels were associated with presence of one allele with less than 28 and the other with more than 33 repeats (16). Spitzer et al., on the contrary, has found no such association when studied a similar but larger cohort (17).

AGG interspersions function as anchor that avoid DNA slippage during DNA replication (18) (19), making the repeats more stable when interrupted with AGGs and hindering the expansion to pathogenic intervals (20). The presence of AGGs decreases instability of premutated alleles, particularly in maternal transmissions. Furthermore Napierala, and co-workers (2005), have demonstrated that the presence of AGG interruptions in the FMR1 repetitive region can influence FXTAS (Fragile X-associated tremor/ataxia syndrome) clinical outcome in male premutation carriers, by weakening the FMR1 mRNA structure (21). The authors observed that transcripts sharing a common AGG pattern acquired similar types of stable secondary structures, irrespective of distinct repeat lengths. AGG pattern has been hypothesized as a cause of the phenotype diversity, observed in premutation carriers. Thus, the study of AGG number and pattern has an important clinical impact in expanded alleles. However, there is currently no information regarding the AGG pattern in normal-sized. The present application discloses the role played in the female ovarian function, by AGG interspersions present in FMR1 alleles showing normal and sub-normal genotypes. Both prior art documents U.S. Pat. No. 9,157,117B2 and US20110020795A1 defined new ranges of CGG repeats on the FMR1 gene relevant to ovarian health: a normal (norm) range of CGGₙ=26-34, a low range of CGGₙ<26 and a high range of CGGₙ>34. However, the inventors of the present patent application as well as others (17,22), could not find any correlation between this FMR1 subgenotypes and hormonal levels or antral follicle counts.

To address this problem, the AGG number and pattern in 50 healthy females is analysed herein. Overall, the results disclosed in the present patent application, confirm the association of the FMR1 CGG repetitive region in the female ovarian function and suggest that the stability of the alleles—determined by AGG number and pattern—is also a determining factor for the ovarian response success.

### SUMMARY

The present application relates to a method of evaluating female reproductive function.

According to the present application the method for evaluating female reproductive function comprises the following steps:
obtaining genomic DNA from a female subject's blood;
measuring the number of triplet CGG repeats on each allele of the FMR1 gene;
determining the AGG interspersions number and pattern;
calculating the allelic score based on a mathematical formula.

In one embodiment the allelic score is calculated according to the following score:

$$Allelic Score = ( {\sum }_{i = 1}^{n} {R}_{i} \times {4}^{i - 1} ) + ( {R}_{n + 1} \times {4}^{n} )$$

Wherein,

Rᵢ is number of CGG repeats before the first AGG interruption of order i (counting from 5′ to 3′);

n is total number of AGG interspersions;

Rₙ₊₁ is the number of CGG repeats after the last AGG interruption.

In one embodiment, the method for evaluating female reproductive function described herein is used in predicting of infertility.

In one embodiment, the method for evaluating female reproductive function described herein is used in the selection of ideal oocyte donor.

In one embodiment, the method for evaluating female reproductive function described herein is used in determining premature ovarian aging predisposition.

The present application has been made in view of the above problems, and that is one object of the present invention to provide a biomarker assay to assess female reproductive function, namely to predict infertility, to assist in the selection of ideal oocyte donors and to diagnose premature ovarian aging predisposition.

### DETAILED DESCRIPTION

The present application relates to a method to assess female reproductive function and ovarian response based on the number of the FMR1 gene CGG repeat and the AGG interspersions number and pattern on each allele. The number of CCG triplets, as well as the AGG number and pattern, is determined by an assay.

Using the mathematical formula disclosed herein, it is possible to calculate an allelic score based on allele size, AGG number and pattern. The allelic score reflects the structure and complexity of the allele. The allelic score is a “signature” reflecting each interspersion pattern. Combining the allelic score of each allele allowed sample distribution into distinct groups: Equivalent pattern group (both alleles have the same number of triplets AGG) and Opposite pattern group (alleles have a different number of triplets) AGG.

1.2. Statistical Analyses

FMR1 genotypes were divided according to the CGG repeat number (23) as “normal” if _{26<}[CGG]_{<34} in both alleles; “normal/high” when 1 allele is in the “normal” range and the other has a _{34<}[CGG]_{<55}; “normal/low” when 1 allele is in the “normal” range and the other has a _{8<}[CGG_{]<26}; “high/low” when 1 allele is in the _{34<}[CGG]_{<55} and the other is _{8<}[CGG]_{<26}; “high/high” when both alleles are in the _{34<}[CGG]_{<55}; “low/low” when both alleles are in the _{8<}[CGG]_{<26}.

Several sets of analyses were carried out: a) parametric statistics and multiple linear regression were calculated using Minitab® statistical software, version 16 (Minitab® Inc., State College, USA). A significance level of 0.05 was considered for all the analyses.

b) Principal component analysis was used to arrange the samples in a multi-dimensional space, using the Canoco for Windows, version 4.5.

Summary of FMR1 genotyping results are shown in Table 1. Data are divided according to FMR1 sub-genotypes previously defined (23).

TABLE 1
Summary of FMR1 genotyping data in
the cohort of 50 samples.

| | | FMR1 | |
|---|---|---|---|
| | CGG repeat | sub-genotypes | |
| Alleles | number | classification | N |
| A1 | _{26 <} CGG _{< 34} | normal | 23 |
| A2 | | | |
| A1 | _{8 <} CGG _{< 25} | low/normal | 17 |
| A2 | _{26 <} CGG _{< 34} | | |
| A1 | _{8 <} CGG _{< 26} | low/high | 4 |
| A2 | _{35 <} CGG _{< 55} | | |
| A1 | _{26 <} CGG _{< 34} | normal/high | 3 |
| A2 | _{35 <} CGG _{< 55} | | |
| A1 | _{8 <} CGG _{< 26} | low/low | 2 |
| A2 | | | |
| A1 | _{34 <} CGG _{< 55} | high/high | 1 |
| A2 | | | |

A1—Allele 1 (smallest in size);
A2—Allele 2 (largest in size);
N—number of samples.

Table 2
Summary of variables used in statistical analysis.

| | | Reference |
|---|---|---|
| N = 50 | | values* |
| Number of antral | 8 ± 4.4 | >6 |
| follicles | | |
| FSH | 5.8 ± 1.7 | <10 mIU/mL |
| LH | 5.9 ± 5.1 | <10 mUI/mL |
| Estradiol | 40.6 ± 24.8 | <60 pg/mL |
| Prolactin | 14.1 ± 6.4 | <25 ng/mL |

Ages ranged from 18 to 33 years (mean±SD=25.4±3.9). Table 2 presents the variables used in our statistical analysis, their mean and standard variation.

An exploratory approach to identify an association between the CGG number and hormonal levels was performed. The six categories of FMR1 sub-genotypes were defined as species and each biochemical parameter (FSH, LH, estradiol and prolactin levels) as supplementary explanatory variants. The values were centered and standardized within Canoco but were not transformed, yielding a biplot correlation. In standardization, all variables were considered equally important regardless of their variability. The biplot in FIG. 1 depicts the association between samples (grouped according with CGG repeat number and corresponding FMR1 sub-genotype) and hormonal levels. The distribution of the samples is determined mainly by estradiol (first axis) and prolactin (second axis). However, the hormonal profile is not able to separate the groups defined by the CGG number. Among the hormones selected for the current study the estradiol alone explained 93.2% of the total variance. Multivariate analysis to project the association between the CGG repeat number and the different species, hindered the individualization of the samples classified by FMR1 sub-genotype. The biplot shows that the hormonal levels are not sufficient to discriminate samples according to the FMR1 sub-genotypes which may be due to the large variability of the hormonal levels observed among the different samples or to the fact that the CGG repeat number and the hormonal levels are independent variables. According to the present application, it is hypothesized that both the length of the CGG tract and the pattern of the AGG interspersions, could play a role in the female reproductive function by a mechanism involving mRNA, similar to that described by Napierala, and co-workers (21). A mathematical formula was designed to score FMR1 alleles according to the CGG number and AGG number and pattern. The score was denominated allelic complexity score value. Using this approach not only the size but also the stability—as determined by the AGG number and pattern—were considered.

$$Allelic Score = ( {\sum }_{i = 1}^{n} {R}_{i} \times {4}^{i - 1} ) + ( {R}_{n + 1} \times {4}^{n} )$$

Rᵢ: number of CGG repeats before the first AGG interruption of order i (counting from 5′ to 3′)

n: total number of AGG interspersions.

Rₙ₊₁: number of CGG repeats after the last AGG interruption.

This mathematical formula simultaneously combines the allelic size and the AGG interspersion number and pattern. The allelic score reflects the structure and complexity of the AGG interspersion pattern.

A clear correlation could not be found between the allelic scores when they are plotted one against the other (FIG. 2).

Nevertheless, when the graph is divided in quadrants centered at an allelic score of 135 (FIG. 3), two distinct patterns emerge: one where a similar AGG interspersion pattern can be observed for both alleles (equivalent group) and a second in which both alleles present a different AGG interspersion pattern (opposite group). The equivalent group includes samples where both alleles share similar number of AGG interruptions (e.g. one or two). The allelic score used to define the quadrants (135 in population analysed in the present application) is based on the fact that:
Alleles are interchangeable, thus associated allelic scores can be positioned either on the X or the Y axis without changing the intrinsic relationship between scores of associated with each allele;
The regression line associated with the equivalent group intercepts the regression line of the opposite group at a point with coordinates (135, 135).

TABLE 3
Distribution according with allelic complexity
groups and FMR1 sub-genotypes

| | N | | N | |
|---|---|---|---|---|
| FMR1 | Equivalent | | Opposite | |
| sub-genotype | group | % | group | % |
| Normal | 15 | 31 | 8 | 16 |
| low/normal | 5 | 10 | 12 | 25 |
| low/high | 1 | 2 | 3 | 6 |
| normal/high | 1 | 2 | 1 | 2 |
| low/low | 2 | 4 | 0 | 0 |
| high/high | 1 | 2 | 0 | 0 |
| TOTAL (N = 49) | 25 | 51 | 24 | 49 |

N—number of samples.

As shown in Table 3, equivalent group is mainly composed by samples carrying alleles in the normal FMR1 sub-genotype. In the opposite group, samples with an FMR1 low/normal sub-genotype are more common. To strengthen confirm the hypothesis that AGG can influence the ovarian function a correlation between the number of antral follicles and the hormonal levels was attained in the equivalent group, using Minitab® 16 statistical software.

This process was initiated by a stepwise regression performed with all quantified hormones. A positive correlation between number of antral follicles, and prolactin and LH levels was obtained. A multiple regression for the number of antral follicles using prolactin and LH as descriptors was performed to establish a statistically significant model (p=0.030) that predicted the number of antral follicles based on the LH and prolactin levels (FIG. 4):

tAFC=3.62+0.523×LH+0.210×PRL

This observation is in line with previous publications that suggest a negative influence of a low FMR1 CGG number on the ovarian reserve, notwithstanding other elements seem to be contributing to this correlation (24).

According to the model disclosed herein, it is possible to theoretically determine the largest number of antral follicles produced combining the levels of prolactin and LH in the group of females that show an equivalent AGG pattern (FIG. 4). These data corroborate that the FMR1 CGG repetitive region has an impact on the female reproductive function and that AGG interspersions can be used to assess the ovarian response success.

Several features are described hereafter that can each be used independently of one another or with any combination of the other features. However, any individual feature might not address any of the problems discussed above or might only address one of the problems discussed above. Some of the problems discussed above might not be fully addressed by any of the features described herein. Although headings are provided, information related to a particular heading, but not found in the section having that heading, may also be found elsewhere in the specification.

### BRIEF DESCRIPTION OF DRAWINGS

The accompanying drawings illustrate various results and embodiments of the present invention and are a part of the specification. The illustrated embodiments are merely examples of the present invention and do not limit the scope of the invention.

FIG. 1 shows a biplot of FMR1 sub-genotypes biochemical results for the 50 female samples.

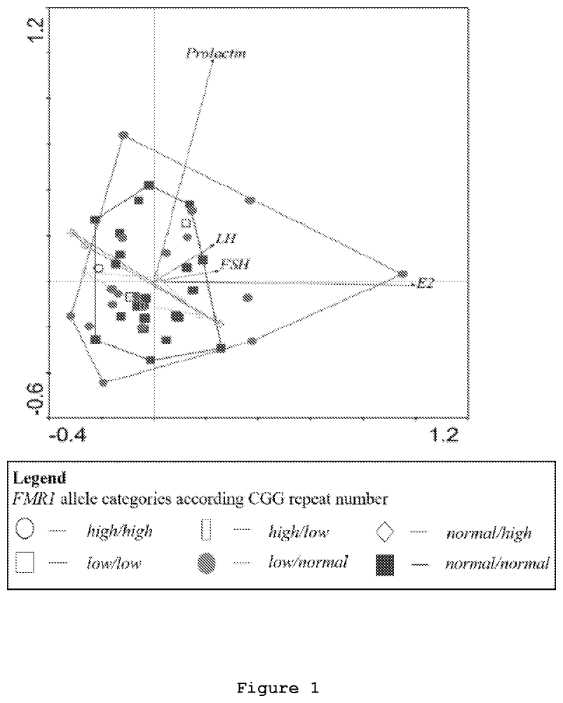

FIG. 2 shows the allelic complexity score value of each sample.

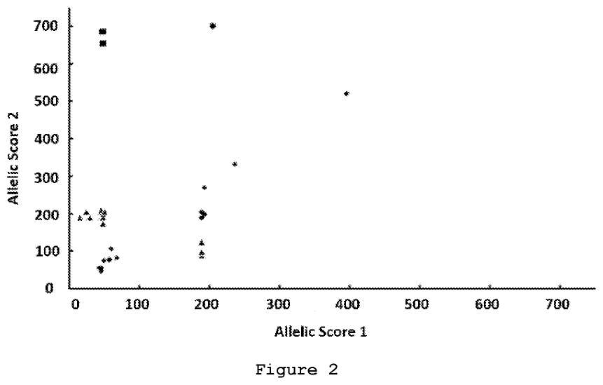

FIG. 3 shows the allelic complexity score value based on the allele size and AGG interruption number and pattern. Samples carrying alleles of equivalent AGG pattern are represented with lozenges and those with an opposite pattern with triangles.

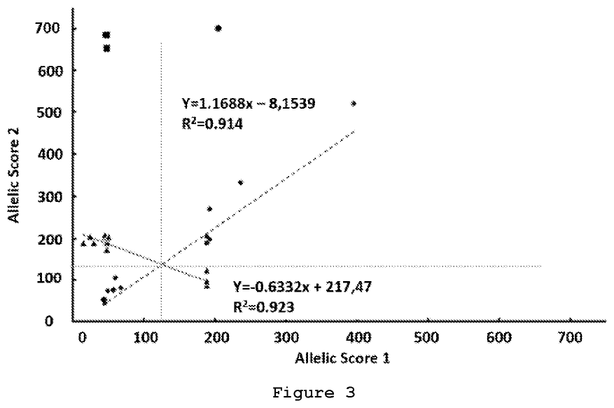

FIG. 4 illustrates an isobologram showing the visual representation of the mathematical formula. Axes show the LH and Prolactin levels. Each color is associated with a specific number of antral follicles. A low number of follicles is represented in black and the maximum in grey. tAFC—Total Antral Follicle.

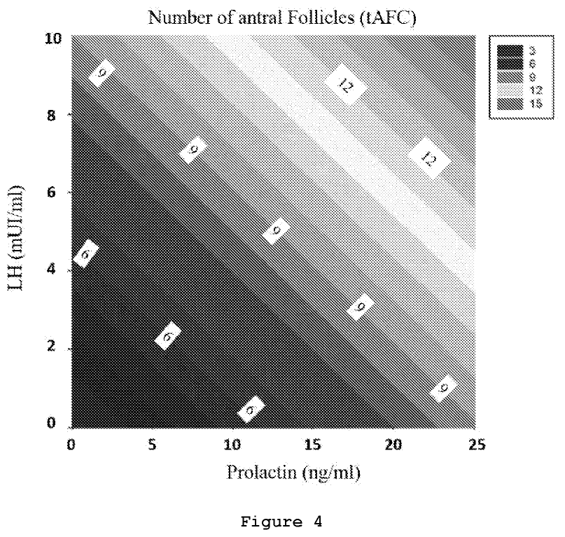

### BEST MODE FOR CARRYING OUT THE INVENTION

Now, preferred embodiments of the present application will be described in detail with reference to the annexed drawings. However, they are not intended to limit the scope of this application.

According to the present application the method for evaluating female reproductive function comprises the following steps:
obtaining genomic DNA from a female subject's blood;
measuring the number of triplet CGG repeats on each allele of the FMR1 gene;
determining the AGG interspersions number and pattern;
calculating the allelic score based on a mathematical formula.

In one embodiment the allelic score is calculated according to the following score:

$$Allelic Score = ( {\sum }_{i = 1}^{n} {R}_{i} \times {4}^{i - 1} ) + ( {R}_{n + 1} \times {4}^{n} )$$

Wherein,

Rᵢ is number of CGG repeats before the first AGG interruption of order i (counting from 5′ to 3′);

n is total number of AGG interspersions;

Rₙ₊₁ is the number of CGG repeats after the last AGG interruption.

In one embodiment, the method for evaluating female reproductive function described herein is used in predicting of infertility.

In one embodiment, the method for evaluating female reproductive function described herein is used in the selection of ideal oocyte donor.

In one embodiment, the method for evaluating female reproductive function described herein is used in determining premature ovarian aging predisposition.

This description is of course not in any way restricted to the forms of implementation presented herein and any person with an average knowledge of the area can provide many possibilities for modification thereof without departing from the general idea as defined by the claims. The preferred forms of implementation described above can obviously be combined with each other. The following claims further define the preferred forms of implementation.

### REFERENCES

1. Tural S, Tekcan A, Kara N, Elbistan M, Guven D, Tasdemir H A. FMR1 gene mutation screening by TP-PCR in patients with premature ovarian failure and fragile X. Gynecol Endocrinol 2014; 3590(3):1-5.
2. Sullivan A K, Marcus M, Epstein M P, Allen E G, Anido A E, Paquin J J, et al. Association of FMR1 repeat size with ovarian dysfunction. Hum Reprod 2005; 20(2):402-12.
3. Murray A, Schoemaker M J, Bennett C E, Ennis S, Macpherson J N, Jones M, et al. Population-based estimates of the prevalence of FMR1 expansion mutations in women with early menopause and primary ovarian insufficiency. Genet Med 2014; 16(1):19-24.
4. Streuli I, Fraisse T, Ibecheole V, Moix I, Morris M A, de Ziegler D. Intermediate and premutation FMR1 alleles in women with occult primary ovarian insufficiency. Fertil Steril 2009; 92(2):464-70.
5. Eslami A, Farahmand K, Totonchi M, Madani T, Asadpour U, Zari Moradi S, et al. FMR1 premutation: not only important in premature ovarian failure but also in diminished ovarian reserve. Hum Fertil 2016; 10(2):1-6.
6. Oostra B A, Willemsen R. A fragile balance: FMR1 expression levels. Hum Mol Genet 2003; 12(2):R249-57.
7. Maia N, Loureiro J R, Oliveira B, Marques I, Santos R, Jorge P, et al. Contraction of fully expanded FMR1 alleles to the normal range: predisposing haplotype or rare events? J Hum Genet 2016; 62(2):1-7.
8. Poon P M K, Chen Q L, Zhong N, Lam S. S, Lai K Y C, Wong C K, et al. AGG interspersion analysis of the FMR1 CGG repeats in mental retardation of unspecific cause. Clin Biochem2006; 39(3):244-8.
9. Schenkel L C, Schwartz C, Skinner C, Rodenhiser D, Ainsworth P, Pare G, et al. Clinical Validation of Fragile X Syndrome Screening by DNA Methylation Array. J Mol Diagnostics 2016; 18(6):834-41.
10. Hoyos L R, Thakur M. Fragile X premutation in women: recognizing the health challenges beyond primary ovarian insufficiency. J Assist Reprod Genet 2017; 34(3):315-23.
11. Saldarriaga W, Tassone F, GonzAlez-Teshima L Y, Forero-Forero J V., Ayala-Zapata S, Hagerman R. Fragile X syndrome. Colomb medica 2014; 45(4):190-8.
12. Wittenberger M D, Hagerman R J, Sherman S L, McConkie-Rosell A, Welt C K, Rebar R W, et al. The FMR1 premutation and reproduction. Fertil Steril 2007; 87(3):456-65.
13. Willemsen R, Levenga J, Oostra B. CGG repeat in the FMR1 gene: Size matters. Clin Genet 2011; 80(3):214-25.
14. Ruth K S, Bennett C E, Schoemaker M J, Weedon M N, Swerdlow A J, Murray A. Length of FMR1 repeat alleles within the normal range does not substantially affect the risk of early menopause. Hum Reprod 2016; 31(10):2396-403.
15. Gleicher N, Yu Y, Himaya E, Barad D H, Weghofer A, Wu Y, et al. Early decline in functional ovarian reserve in young women with low (CGGn<26) FMR1 gene alleles. Transl Res 2015; 166(5):502-7.
16. Gleicher N, Weghofer A, Oktay K, Barad D H. Revelance of triple CGG repeats in the FMR1 gene to ovarian reserve. Acta Obstet Gynecol Scand 2009; 88(9):1024-30.
17. Spitzer L T, Johnstone E B, Huddleston H G, Cedars L M, Davis G, Fujimoto V. FMR1 Repeats and Ovarian Reserve: CGG Repeat Number does not Influence Antral Follicle Count. J Fertil Vitr 2012; 02(03):10-3.
18. Pearson C E, Eichler E E, Lorenzetti D, Kramer S F, Zoghbi H Y, Nelson D L, et al. Interruptions in the triplet repeats of SCAl and FRAXA reduce the propensity and complexity of slipped strand DNA (S-DNA) formation. Biochemistry 1998; 37(8):2701-8.
19. McGinty R J, Mirkin S M. Cis- and Trans-Modifiers of Repeat Expansions: Blending Model Systems with Human Genetics. Trends Genet 2018; 34(6):448-65.
20. Yrigollen C M, Durbin-Johnson B, Gane L, Nelson D L, Hagerman R, Hagerman P J, et al. AGG interruptions within the maternal FMR1 gene reduce the risk of offspring with fragile X syndrome. Genet Med 2012; 14(8):729-36.
21. Napierala M, Michalowski D, de Mezer M, Krzyzosiak W J. Facile F M R1 mRNA structure regulation by interruptions in CGG repeats. Nucleic Acids Res 2005; 33(2):451-63.
22. Maslow B S L, Davis S, Engmann L, Nulsen J C, Benadiva C A. Correlation of normal-range FMR1 repeat length or genotypes and reproductive parameters. J Assist Reprod Genet 2016; 33(9):1149-55.
23. Gleicher N, Kushnir V A, Weghofer A, Barad D H. How the FMR1 gene became relevant to female fertility and reproductive medicine. Front Genet 2014; 5(284):1-5.
24. Kline J K, Kinney A M, Levin B, Brown S A, Hadd A G, Warburton D. Intermediate CGG repeat length at the FMR1 locus is not associated with hormonal indicators of ovarian age. J North Am Menopause Soc [Internet]2014; 21(7):740-8.

1. A method of evaluating female reproductive function comprising the following steps:
obtaining genomic DNA from a female subject's blood;
measuring the number of triplet CGG repeats on each allele of the FMR1 gene;
determining the AGG interspersions number, the number CGG repeats before the first AGG interruption and the number of CGG repeats after the last AGG interruption;
calculating the allelic score according to the following mathematical formula:
$$Allelic Score = ( {\sum }_{i = 1}^{n} {R}_{i} \times {4}^{i - 1} ) + ( {R}_{n + 1} \times {4}^{n} )$$
wherein,
Rᵢ is number of CGG repeats before the first AGG interruption of order i (counting from 5′ to 3′);
n is total number of AGG interspersions;
Rₙ₊₁ is the number of CGG repeats after the last AGG interruption;
wherein subjects with an allelic score similar for both alleles have a better reproductive performance.

2. A method for prediction of infertility comprising carrying out the steps of the method according to claim 1.

3. A method for selection of ideal oocyte donors comprising carrying out the steps of the method according to claim 1.

4. A method for determination of premature ovarian aging predisposition comprising carrying out the steps of the method according to claim 1.
